(19) United States
(12) Patent Application Publication
(10) Pub. No.: US 2025/0264565 A1
(43) Pub. Date: Aug. 21, 2025

(54) ECHO-SHIFTED ECHO-PLANAR IMAGING WITH SIMULTANEOUS BLIP-UP AND BLIP-DOWN ACQUISITIONS FOR CORRECTING GEOMETRIC DISTORTION

(71) Applicant: THE BOARD OF TRUSTEES OF THE UNIVERSITY OF ILLINOIS, URBANA, IL (US)

(72) Inventors: Xiaohong Joe Zhou, Naperville, IL (US); Kaibao Sun, Chicago, IL (US)

(73) Assignee: THE BOARD OF TRUSTEES OF THE UNIVERSITY OF ILLINOIS, URBANA, IL (US)

(21) Appl. No.: 18/857,226

(22) PCT Filed: Apr. 18, 2023
(86) PCT No.: PCT/US2023/018940

§ 371 Date: Oct. 16, 2024

Related U.S. Application Data
(60) Provisional application No. 63/332,260, filed on Apr. 18, 2022.

(51) Int. Cl. G01R 33/565; A61B 5/055; G01R 33/56; G01R 33/561

(52) U.S. Cl. CPC G01R 33/56554; G01R 33/5608; G01R 33/5616; A61B 5/055

(57) ABSTRACT

The present disclosure provides an example method for using an MRI system electrically coupled to a computing device. The method includes generating, via the MRI system, an echo-shifted echo-planar imaging with blip up/down acquisition (“esEPI-BUDA”) pulse sequence including a first radiofrequency (“RF”) pulse and a second RF pulse, the first RF pulse followed by a first echo-train that is interleaved with the first and the second RF pulses, and the second RF pulse followed by a second echo-train such that the first and the second echo-trains have opposite phase-encoding blip gradient polarities to traverse echo planar imaging (“EPI”) k-space in a reversed order. In response to the pulse sequence being generated, the MRI system acquires two k-space datasets within a single shot and corrects image distortion, via the MRI system, based on the two acquired k-space datasets.

### CROSS-REFERENCE TO RELATED APPLICATION

This international PCT application claims the benefit of the filing date of U.S. Provisional Patent Application No. 63/332,260, filed on Apr. 18, 2022, that is hereby incorporated by reference in its entirety.

### STATEMENT OF GOVERNMENTAL INTEREST

This invention was made with awards 5R01EB026716 and 1S10RR028898 from the National Institute of Health (NIH). The government has certain rights in the invention.

### BACKGROUND

Blood oxygenation level-dependent (BOLD) functional MRI (fMRI) is a primary technique for mapping neural activity of the human brain. Human brain mapping using MRI is typically performed using a set of two-dimensional axial slices. However, three-dimensional (3D) acquisitions using gradient-echo echo-planar imaging (GRE-EPI) are gaining momentum due to their fast acquisition speed, robustness against motion, whole-brain spatial coverage, and high signal-to-noise ratio (SNR) per unit time. In 3D GRE-EPI, conventional phase-encoding with a stepping gradient is employed in one spatial dimension (e.g., the z-axis or the “slice” direction), while EPI-type phase encoding with blip gradients is applied to the second spatial dimension (e.g., the y-axis or the phase direction) and EPI-type bipolar readout gradient is used to spatially encode the third dimension. However, since the sampling bandwidth is narrow (e.g., <2 kHz) in the blipped phase-encoding direction, severe geometric distortions can occur in the presence of magnetic field inhomogeneities and other off-resonance effects. If the associated perturbing field gradient has the same polarity as the blip phase-encoding gradient, then the images are stretched along the phase-encoding direction. Conversely, if the gradient polarities are opposite, then the images are compressed.

Several distortion correction techniques have been proposed for EPI images over the past decades. One approach is to correct distortions in the image domain using a B₀-field map, which is obtained from a double-echo gradient echo (“GRE”) image. The method can effectively correct signal-stretching artifacts in the images. However, an EPI acquisition with only one phase-encoding polarity does not provide sufficient information to correct for signal pile-up in regions with substantial off-resonance effects, where signals from different locations are compressed into a single pixel and hence their spatial information is lost. To address this issue, image-domain registration based on two EPI data sets with opposite phase-encoding directions (e.g., TOPUP in FSL) have been developed, and adopted in several large-scale neuroimaging studies. Nonetheless, the registration errors may fail distortion correction when the image SNR is poor, especially in the anterior temporal lobe and orbitofrontal cortex. This issue has been addressed using a technique known as BUDA (blip-up/down acquisition). BUDA is based on a forward model that links the distortion-free image to the corrupted raw k-space data. This allows distortion-corrected images to be reconstructed from the k-space data acquired with blip-up and blip-down phase-encoding gradients by solving the corresponding inverse problem with Hankel structured low-rank constraint. In previous studies, BUDA has been successfully applied to susceptibility, diffusion and relaxation-weighted imaging and demonstrated its robustness in geometric distortion correction for two-dimensional (“2D”) and 3D EPI.

Despite its excellent performance, BUDA requires two separate acquisitions (or shots) with blip-up and blip-down, respectively, which compromises the imaging efficiency. This can be particularly problematic for fMRI because the need for two acquisitions halves the temporal resolution for resolving BOLD signal changes. Moreover, the longer scan times can also impose a physiological burden to the subject and increase vulnerability to motion. Very recently, an echo-shifting technique was employed to incorporate two EPI echo-trains with the same phase-encoding gradient polarity into a single sequence to increase the time efficiency for 2D EPI. This approach, however, is subject to a serious SNR loss caused by large flip angles needed in 2D multi-slice acquisitions. For example, only ˜50% SNR can be retained under the optimal condition when compared to conventional 2D EPI with a 90° flip angle.

### SUMMARY

Systems, methods, and computer readable mediums for correcting geometric distortion in echo-planar magnetic resonance imaging are set forth herein. The echo planar imaging (“EPI”) sequence is widely used in magnetic resonance imaging (“MRI”) due to its fast acquisition speed and robustness against motion. This method, however, is subject to geometric distortion caused by various off-resonance effects, including magnetic field inhomogeneities and magnetic susceptibility variations. Although the issue can be addressed by acquiring two k-space data sets with the opposite phase-encoding gradient polarity (i.e., blip-up/-down acquisition or BUDA) followed by combining the datasets to estimate a field map for distortion correction, a disadvantage is the doubled scan time due to the need of the two separately acquired datasets. Exemplary implementations disclosed herein integrate the two acquisitions into a single acquisition (or single shot) by using a novel echo-shifting technique. This method or technique is referred to herein as “Echo-shifted EPI with blip-up and -down acquisitions (‘esEPI-BUDA’).”

In accordance with the principles disclosed herein, esEPI-BUDA consists of two radiofrequency (“RF”) pulses in one repetition time (or one shot), followed by two successive echo-trains with polarity-reversed phase-encoding gradients. A set of “echo-shifting gradient pulses” can be employed to allow the two echo-trains to acquire the signals from the first and second RF pulses, respectively. Thus, esEPI-BUDA can beneficially halve the total scan time of the conventional BUDA method and minimize inconsistent phase variations due to subject motion between the two acquisitions. By integrating the two acquisitions into one shot followed by joint image reconstruction, the exemplary methods herein can produce distortion-free images without increasing the scan times and without being subject to patient motion. This exemplary technique is demonstrated herein on phantoms and human subjects.

In a first aspect of the disclosure, an example method for using an MRI system electrically coupled to a computing device is disclosed. The method includes (a) generating, via the MRI system, an echo-shifted echo-planar imaging with blip up/down acquisition (“esEPI-BUDA”) pulse sequence comprising a first radiofrequency (“RF”) pulse and a second RF pulse, the first RF pulse followed by a first echo-train that is interleaved with the first and the second RF pulses, and the second RF pulse followed by a second echo-train such that the first and the second echo-trains have opposite phase-encoding blip gradient polarities to traverse echo planar imaging (“EPI”) k-space in a reversed order, (b) in response to the pulse sequence being generated, the MRI system acquiring two k-space datasets within a single shot, and (c) correcting image distortion, via the MRI system, based on the two acquired k-space datasets.

In a second aspect of the disclosure, an example non-transitory computer-readable medium is disclosed. The computer-readable medium has stored thereon program instructions that upon execution by a processor, cause performance of a set of steps including (a) an MRI system generating an echo-shifted echo-planar imaging with blip up/down acquisition (“esEPI-BUDA”) pulse sequence comprising a first radiofrequency (“RF”) pulse and a second RF pulse, the first RF pulse followed by a first echo-train that is interleaved with the first and the second RF pulses, and the second RF pulse followed by a second echo-train such that the first and the second echo-trains have opposite phase-encoding blip gradient polarities to traverse echo planar imaging (“EPI”) k-space in a reversed order, (b) in response to the pulse sequence being generated, the MRI system acquiring two k-space datasets within a single shot, and (c) the MRI system correcting image distortion based on the two acquired k-space datasets.

The features, functions, and advantages that have been discussed can be achieved independently in various examples or may be combined in yet other examples further details of which can be seen with reference to the following description and drawings.

### BRIEF DESCRIPTION OF THE DRAWINGS

FIG. 1 is a functional block diagram of an MRI system electrically coupled to a computing device, according to one example implementation;

FIG. 2 depicts a block diagram of a computing device and a computer network, according to an example implementation:

FIG. 3 shows a flowchart of a method for using the MRI system, according to an example implementation:

FIGS. 4A-B depict a schematic illustrating 3D echo-shifted EPI with blip-up and blip-down acquisitions (“esEPI-BUDA”) in a single repetition time (“TR”) (FIG. 4A), and the corresponding two k-space trajectories (FIG. 4B). Echo-shifting gradients (with areas of (G−A), −G, and G as indicated in the figure) are applied along the slab-selection direction (G_{z}) to select the signals for the two echo-train acquisitions (black on the lefthand side and gray on the righthand side) from the first and second RF pulses, respectively. A small gradient with ½ phase-encoding blip area (G_{y}) is played out prior to the second echo-train so that the two k-space trajectories are interleaved, according to one example implementation:

FIG. 5 depicts the image reconstruction steps involved in esEPI-BUDA by using the human brain as an example. Each under-sampled echo-train dataset (i.e., Echo-train 1 with blip-up acquisition and Echo-train 2 with blip-down acquisition) first underwent 3D SENSE reconstruction individually, followed by TOPUP in FSL to estimate a field map E. The field map was subsequently incorporated to jointly reconstruct the data from both echo-trains with Hankel structured low-rank regularization for geometric distortion correction, according to one example implementation:

FIG. 6 depicts representative images of a slice selected from the 3D datasets of DQA phantom acquired using 3D fully-sampled EPI with separate blip-up (A) and blip-down (B) acquisitions, 3D EPI TOPUP (C), 3D esEPI-BUDA (SENSE EPI in Echo-train 1 (D); SENSE EPI in Echo-train 2 (E); esEPI-BUDA (F)), and 3D SPGR (G). Using the horizontal black block (low-signal area indicated by the arrow in (G) as a fiducial mark, the images in (A) and (B) exhibited excessive image distortion that was reduced in (D) and (E). Image distortion was substantially corrected in (C) and (F). Image (C) shows that the geometric distortion was corrected by image registration of two 3D fully-sampled EPI (A) and (B) images using TOPUP in FSL. Image (F) shows that 3D esEPI-BUDA produced distortion-corrected images by jointly reconstructing two interleaved k-space datasets with reverse filling trajectories. Using the 3D SPGR image as a reference, the esEPI-BUDA image exhibited a higher degree of similarity with a SSIM of 0.91 and a lower NRMSE of 0.06, than the 3D EPI TOPUP image with a SSIM of 0.87 and a NRMSE of 0.08, according to one example implementation:

FIG. 7A depicts a set of 120 B₀ maps in a slice randomly selected from the 3D datasets of the human brain, covering a total time span of 4 min and 48 sec with a temporal resolution of 2.4 sec:

FIG. 7B depicts the dynamic B₀ evolutions at the frontal (top box of FIG. 7A) and occipital (bottom box of FIG. 7B) lobes, according to one example implementation:

FIG. 7C depicts the corresponding spatial Δd dislocation caused by B₀. As shown in FIGS. 7B-7C, the maximal B₀ shifts during the process were approximately 6.2 Hz and 4.7 Hz in the two brain regions, corresponding to the spatial dislocations of 1.95 mm and 1.47 mm, respectively, according to one example implementation:

FIGS. 8A-C depict representative whole-brain 3D images from a healthy human subject (SENSE EPI in Echo-train 1 (FIG. 8A), SENSE EPI in Echo-train 2 (FIG. 8B), esEPI-BUDA (FIG. 8C)) obtained in one volume TR using the 3D esEPI-BUDA sequence. Compared with the SENSE reconstruction using the blip-up or blip-down data, esEPI-BUDA image reconstruction allowed for a joint reconstruction of blip-up and blip-down data and produced improved image quality with the least image distortion, particularly in the areas with large magnetic susceptibility variations, such as the frontal lobe, according to one example implementation:

FIG. 9 depicts representative images of a slice with a voxel size of 3.1×3.1×4 mm³ randomly selected from the 3D datasets of the human brain. Images (A) and (B) were acquired using 3D fully-sampled EPI with separate blip-up (A) and blip-down (B) acquisitions. Image (C) depicts an image reconstructed from images (A) and (B) by using 3D EPI TOPUP. Images (D) and (E) were reconstructed from the first and second echo train of the esEPI-BUDA sequence, respectively. Image (F) shows the resultant esEPI-BUDA image reconstructed from both echo trains. Image (G) displays a conventional 3D SPGR image. Using the 3D SPGR image with a short TE (1.4 ms) as a reference, the images in (A) and (B) exhibited excessive image distortion, which was reduced in images (D) and (E). Image distortion was effectively corrected in images (C) and (F), according to one example implementation:

FIG. 10 depicts representative visual fMRI activation maps of 3D fully sampled EPI with separated blip-up (Row A) and blip-down (Row B) acquisitions, the corresponding 3D EPI TOPUP (Row C), and 3D esEPI-BUDA (Row D), overlaid onto the T1-weighted images that are co-registered with 3D SPGR images. The activation maps co-register better with brain parenchyma in the T1-weighted structure images for 3D esEPI-BUDA, compared with conventional 3D fully-sampled EPI and the corresponding 3D EPI TOPUP images. These functional activation maps were rendered in color in the priority document and more fully depict the contrast between the images, according to one example implementation:

FIGS. 11A-D depict averaged time course with standard deviations (across the six subjects) for the images from 3D fully sampled EPI with separate blip-up (FIG. 8A) and blip-down (FIG. 11B) acquisitions, the corresponding 3D EPI TOPUP (FIG. 11C), and 3D esEPI-BUDA (FIG. 11D). 3D esEPI-BUDA and 3D fully-sampled EPI produced similar BOLD signal changes (˜3%), which was higher than 3D EPI TOPUP images (˜2%). The decrease of signal change for 3D EPI TOPUP images may be caused by the image interpolation in the TOPUP tool of FSL. The black bars represent the time period for the visual stimulus, which was 24 s, according to one example implementation:

FIG. 12 depicts the image reconstruction steps involved in BUDA using the human brain as an example. A field map estimated from separate blip-up and blip-down reconstructions is incorporated into a parallel imaging model to jointly reconstruct two datasets with low-rank regularization for geometric distortion correction. Quantitative measurement of SNR in a homogeneous region of white matter revealed an approximately 13.5% increase in the 3D BUDA image (SNR=14.3) compared with the corresponding 3D SENSE images (SNR=12.6), according to one example implementation:

FIG. 13 depicts a set of representative whole-brain 3D images obtained in one volume TR using 3D esEPI-BUDA. Minimal geometric distortions are seen in the brain areas typically susceptible to distortion, according to one example implementation:

FIGS. 14A-D each depict a representative image of a slice selected from the 3D datasets of human brain acquired using 3D fully-sampled EPI with separately acquired blip-up (FIG. 14A) and blip-down (FIG. 14B) datasets, 3D esEPI-BUDA (FIG. 14C), and 3D T1-weighted GRE (FIG. 14D). As shown, 3D esEPI-BUDA (FIG. 14C) produced images with considerably reduced geometric distortion (e.g., in the frontal lobe), when compared to the 3D fully-sampled EPI results with separately acquired blip-up and blip-down datasets (FIGS. 14A-14B). In all comparisons, the 3D T1-weighted GRE image (FIG. 14D) was used as a reference. The horizontal lines serve as a fiducial mark. Note that the image brightness is intentionally exaggerated to facilitate the comparisons, according to one example implementation:

FIG. 15A depicts visual fMRI activation maps of 3D esEPI-BUDA (first row), compared with 3D fully-sampled EPI with blip-up (second row) and blip-down (third row) acquisitions, according to one example implementation; and

FIG. 15B S % depicts the corresponding time courses over 4 min and 48 s for the fMRI activation maps of FIG. 15A that illustrate comparable BOLD signal changes of approximately 4.0%, according to one example implementation.

The drawings are for the purpose of illustrating examples, but it is understood that the disclosure is not limited to the arrangements and instrumentalities shown in the drawings.

### DETAILED DESCRIPTION

### I. Overview

The disclosed exemplary methods-esEPI-BUDA-advantageously integrate the blip-up and blip-down acquisitions into a single repetition time (“TR”) or shot. Two interleaved k-space datasets with reversed k-space filling trajectories are acquired conjointly in one shot, followed by BUDA reconstruction to produce a distortion-corrected image. The esEPI-BUDA technique, method, systems, and computer-readable mediums are demonstrated in phantoms and the healthy human brain for functional activity mapping.

The disclosed systems, methods, and computer readable mediums can beneficially integrate blip-up and blip-down acquisitions in one shot, avoid shot-to-shot phase variations, eliminate inter-shot motion sensitivity, and reduce the scan times. These features can benefit clinical and research use of MRI on human subjects, as well as animals.

### II. Example Architecture

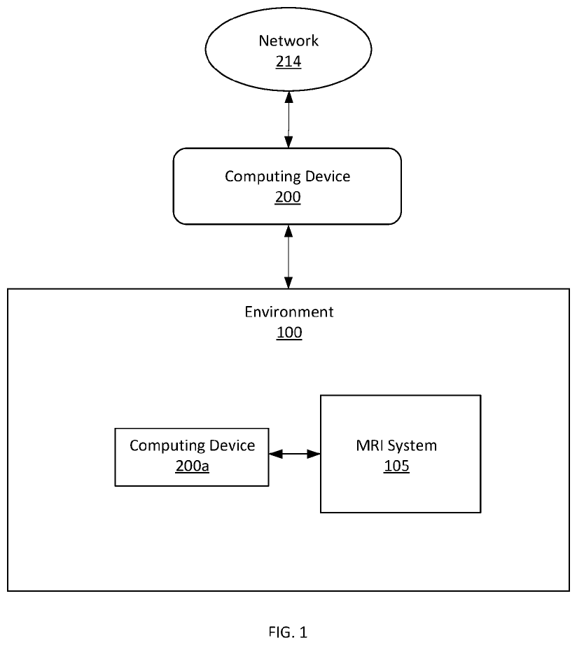

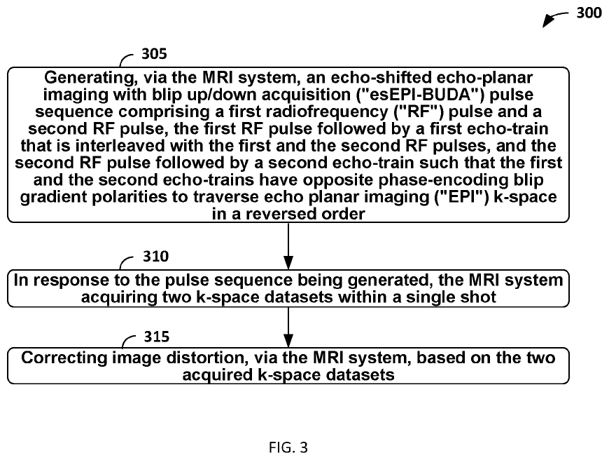

FIG. 1 is a block diagram showing an operating environment 100 that includes or involves, for example, an MRI system 105 described below. Method 300 shown in FIG. 3 and described below is an example of a method that can be implemented within this operating environment 100.

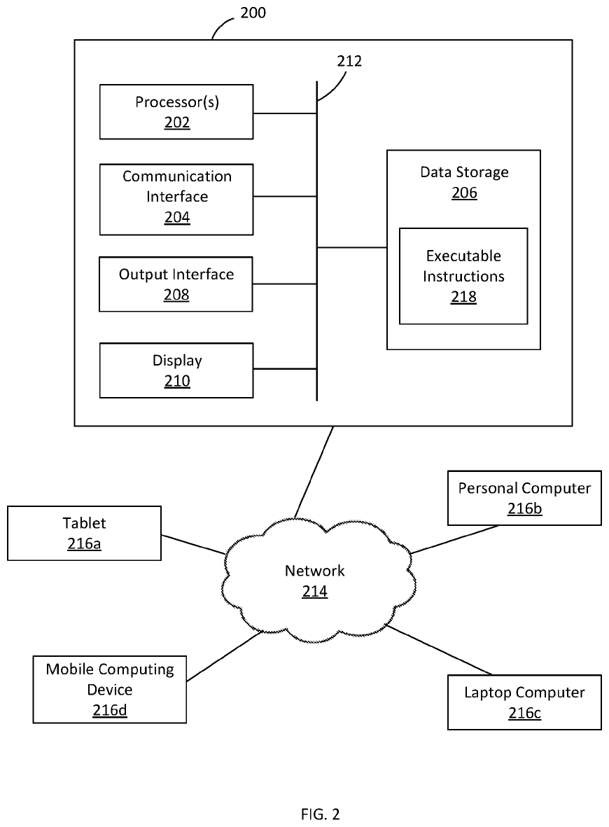

FIG. 2 is a block diagram illustrating an example of a computing device 200, according to an example implementation, that is configured to interface with operating environment 100, either directly or indirectly. The computing device 200 may be used to perform functions of the method shown in FIG. 3 and described below. In particular, computing device 200 can be configured to perform one or more functions, including integrating blip-up and blip-down acquisitions into a single shot and utilizing BUDA reconstruction to produce a distortion-corrected image, for example. The computing device 200 has a processor(s) 202, and also a communication interface 204, data storage 206, an output interface 208, and a display 210 each connected to a communication bus 212. The computing device 200 may also include hardware to enable communication within the computing device 200 and between the computing device 200 and other devices (e.g., not shown). The hardware may include transmitters, receivers, and antennas, for example.

The communication interface 204 may be a wireless interface and/or one or more wired interfaces that allow for both short-range communication and long-range communication to one or more networks 214 or to one or more remote computing devices 216 (e.g., a tablet 216a, a personal computer 216b, a laptop computer 216c and a mobile computing device 216d, for example). Such wireless interfaces may provide for communication under one or more wireless communication protocols, such as Bluetooth. Wi-Fi (e.g., an institute of electrical and electronic engineers (IEEE) 802.11 protocol), Long-Term Evolution (LTE), cellular communications, near-field communication (NFC), and/or other wireless communication protocols. Such wired interfaces may include Ethernet interface, a Universal Serial Bus (USB) interface, or similar interface to communicate via a wire, a twisted pair of wires, a coaxial cable, an optical link, a fiber-optic link, or other physical connection to a wired network. Thus, the communication interface 204 may be configured to receive input data from one or more devices and may also be configured to send output data to other devices.

The communication interface 204 may also include a user-input device, such as a keyboard, a keypad, a touch screen, a touch pad, a computer mouse, a track ball and/or other similar devices, for example.

The data storage 206 may include or take the form of one or more computer-readable storage media that can be read or accessed by the processor(s) 202. The computer-readable storage media can include volatile and/or non-volatile storage components, such as optical, magnetic, organic or other memory or disc storage, which can be integrated in whole or in part with the processor(s) 202. The data storage 206 is considered non-transitory computer readable media. In some examples, the data storage 206 can be implemented using a single physical device (e.g., one optical, magnetic, organic or other memory or disc storage unit), while in other examples, the data storage 206 can be implemented using two or more physical devices.

The data storage 206 thus is a non-transitory computer readable storage medium, and executable instructions 218 are stored thereon. The instructions 218 include computer executable code. When the instructions 218 are executed by the processor(s) 202, the processor(s) 202 are caused to perform functions.

The processor(s) 202 may be a general-purpose processor or a special purpose processor (e.g., digital signal processors, application specific integrated circuits, etc.). The processor(s) 202 may receive inputs from the communication interface 204 and process the inputs to generate outputs that are stored in the data storage 206 and output to the display 210. The processor(s) 202 can be configured to execute the executable instructions 218 (e.g., computer-readable program instructions) that are stored in the data storage 206 and are executable to provide the functionality of the computing device 200 described herein.

The output interface 208 outputs information to the display 210 or to other components as well. Thus, the output interface 208 may be similar to the communication interface 204 and can be a wireless interface (e.g., transmitter) or a wired interface as well. The output interface 208 may send commands to one or more controllable devices, for example.

The computing device 200 shown in FIG. 2 may also be representative of a local computing device 200a in operating environment 100, for example, in communication with and electrically coupled to the MRI system 105. This local computing device 200a may perform one or more of the steps of the method 300 described below, may receive input from a user and/or may send image data and user input to computing device 200 to perform all or some of the steps of method 300.

FIG. 3 shows a flowchart of example method 300 for an MRI system to generate an echo-shifted echo-planar imaging with blip up/down acquisition (“esEPI-BUDA”) pulse sequence with RF pulses in a single shot and to acquire two k-space datasets within the single shot and to correct image distortion, according to an example implementation. Method 300 is an example method that could be used with the computing device 200 of FIG. 2, for example. In some instances, components of MRI system 105 may be configured to perform the functions such that the components are configured and structured with hardware and/or software to enable such performance. Components of the devices and/or systems may be arranged to be adapted to, capable of, or suited for performing the functions, such as when operated in a specific manner. Method 300 may include one or more operations, functions, or actions as illustrated by one or more of blocks 305-315. Although the blocks are illustrated in a sequential order, some of these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of the present examples. In this regard, each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time such as register memory, processor cache and Random Access Memory (RAM). The computer readable medium may also include non-transitory media, such as secondary or persistent long-term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block in FIG. 3, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

### III. Example Methods

The following method 300 may include one or more operations, functions, or actions as illustrated by one or more of blocks 305-315. Although the blocks are illustrated in a sequential order, these blocks may also be performed in parallel, and/or in a different order than those described herein. Also, the various blocks may be combined into fewer blocks, divided into additional blocks, and/or removed based upon the desired implementation. Alternative implementations are included within the scope of the examples of the present disclosure in which functions may be executed out of order from that shown or discussed, including substantially concurrent or in reverse order, depending on the functionality involved, as would be understood by those reasonably skilled in the art.

Referring now to FIG. 3, FIG. 3 shows a flowchart of an example method 300 for using the MRI system 105 electrically coupled to a computing device 200, according to an example implementation. As used herein, “electrically coupled” refers to coupling using a conductor, such as a wire or a conductible trace, as well as inductive, magnetic, and wireless couplings. The computing device 200 may be local or remote to the MRI system 105.

Method 300 includes, at block 305, the MRI system 105 generating an echo-shifted echo-planar imaging with blip up/down acquisition (“esEPI-BUDA”) pulse sequence. The esEPI-BUDA pulse sequence includes a first radiofrequency (“RF”) pulse and a second RF pulse, the first RF pulse followed by a first echo-train that is interleaved with the first and the second RF pulses, and the second RF pulse followed by a second echo-train such that the first and the second echo-trains have opposite phase-encoding blip gradient polarities to traverse echo planar imaging (“EPI”) k-space in a reversed order. Then, at block 310, in response to the pulse sequence being generated, the MRI system 105 acquires two k-space datasets within a single shot (i.e., repetition time, or TR). Next, at block 315, the MRI system 105 corrects image distortion based on the two acquired k-space datasets.

In one optional implementation, the two k-space datasets acquired in a single shot (or TR) may be configured to eliminate or substantially reduce inter-shot phase errors caused by MRI system imperfection, subject motion, and/or other factors. In various example implementations, correcting image distortion may conducted through joint reconstruction or separately followed by geometric corrections using an algorithm, such as EPI TOPUP. In a further optional implementation, the computing device 200 is an MRI reconstruction processor of the MRI system 105 that conducts joint image reconstruction.

In one optional implementation, the first RF pulse has a flip angle of a and the second RF pulse has a flip angle of β, with α and β satisfying the following condition:

$$sin α \cdot {cos}^{2} ( β / 2 ) = cos α \cdot sin β .$$

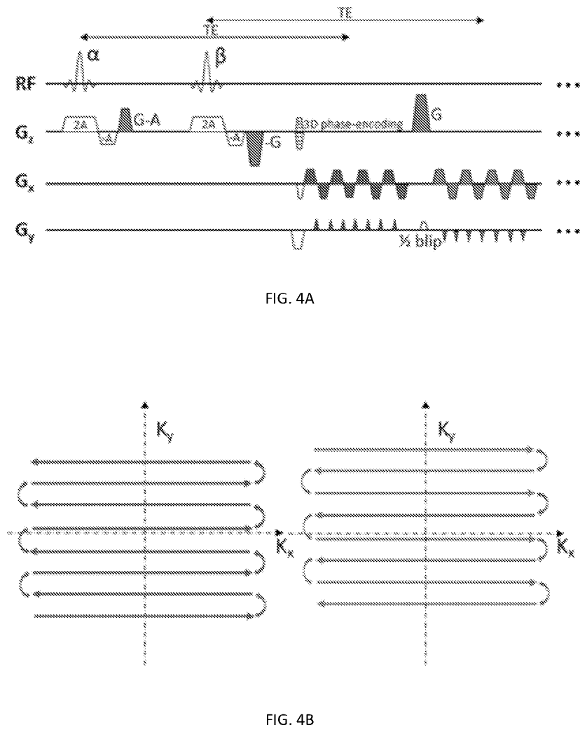

In one optional implementation, generating the esEPI-BUDA pulse sequence further includes the MRI system 105 generating a plurality of echo-shifting gradients applied along a direction perpendicular to the imaging plane. In a further optional implementation, generating the plurality of echo-shifting gradients includes the MRI system 105 generating a first echo-shifting gradient with an area of G′ and thereby dephasing transverse magnetization from the first RF pulse, where A is the absolute value of the slice-refocusing gradient area (or one half of the slice-selection gradient area as shown in FIG. 4A) and G is the absolute value of the nominal dephasing/rephasing gradient area as detailed thereafter. After the transverse magnetization is dephased, the MRI system generates the second RF pulse and thereby excites the stored longitudinal magnetization. After the second RF pulse is generated, the MRI system 105 generates the second echo-shifting gradient with an area of −G and thereby dephases transverse magnetization from the second RF pulse and rephases a signal produced by the first RF pulse. And after the signal produced by the first RF pulse is acquired, generating, via the MRI system, the third echo-shifting gradient with an area of G and thereby dephasing transverse magnetization from the first RF pulse and rephasing a signal produced by the second RF pulse. In a further optional implementation, G′=G−A with A being the absolute value of the area of a slice-refocusing gradient associate with the first or the second RF pulse.

In one optional implementation the first echo-train with blip-up phase-encoding acquires the rephased signal produced by the first RF pulse.

In yet another optional implementation, generating the plurality of echo-shifting gradients further includes, after the first echo-train is generated, the MRI system generating a third echo-shifting gradient with an area of G and thereby rephasing a signal produced by the second RF pulse and dephasing the signal produced by the first RF pulse.

In one optional implementation, the second echo-train with blip-down phase-encoding acquires the rephased signal produced by the second RF pulse.

In one optional implementation, the esEPI-BUDA pulse sequence further includes, after acquiring the rephased signal produced by the first RF pulse and before acquiring the rephased signal produced by the second RF pulse, the MRI system 105 generating a gradient having one half (½) of an individual phase-encoding blip gradient area (G_{y}).

In one optional implementation, the first and second RF pulses and their associated first and second EPI echo-trains and echo-shifting gradients are extended to a plural number greater than two.

In another optional implementation, the esEPI-BUDA pulse sequence is applied for two-dimensional or three-dimensional functional MRI, diffusion MRI, perfusion MRI, and/or other MRI applications where echo-train-based echo planar imaging acquisitions are employed.

In one optional implementation, the MRI system under-samples k-space data from the first echo-train and the second echo-train and thereby shortens a length of each of the first echo-train and the second echo-train.

In one optional implementation, conducting joint image reconstruction based on the two acquired k-space datasets further includes the MRI system 105 generating dynamic maps of a main magnetic field (i.e., B₀ field). And the MRI system incorporating the dynamic maps of a main magnetic field into a forward joint parallel imaging reconstruction model with Hankel structured low-rank constraints and thereby correcting the image geometric distortion.

In another optional implementation, conducting joint image reconstruction based on the two acquired k-space datasets further includes the MRI system 105 combining the two acquired k-space datasets and thereby improving the image signal-to-noise ratio.

### IV. Non-Transitory Computer-Readable Mediums

As discussed above, a non-transitory computer-readable medium having stored thereon program instructions that upon execution by an MRI system 105 electrically coupled to a computing device 200 may be utilized to cause performance of any of functions of the foregoing methods.

As one example, a non-transitory computer-readable medium having stored thereon program instructions that upon execution by a processor, cause performance of a set of steps includes an MRI system 105 generating an echo-shifted echo-planar imaging with blip up/down acquisition (“esEPI-BUDA”) pulse sequence. The esEPI-BUDA pulse sequence includes a first radiofrequency (“RF”) pulse and a second RF pulse, the first RF pulse followed by a first echo-train that is interleaved with the first and the second RF pulses, and the second RF pulse followed by a second echo-train such that the first and the second echo-trains have opposite phase-encoding blip gradient polarities to traverse echo planar imaging (“EPI”) k-space in a reversed order. In response to the pulse sequence being generated, the MRI system 105 acquires two k-space datasets within a single shot. The MRI system 105 then corrects image distortion based on the two acquired k-space datasets. In various example implementations, correcting image distortion may be conducted through joint reconstruction or separately followed by geometric corrections using an algorithm, such as EPI TOPUP.

In one optional implementation, the MRI system 105 generating the esEPI-BUDA pulse sequence further includes the MRI system 105 generating a plurality of echo-shifting gradients applied along a direction perpendicular to the imaging plane.

In another optional implementation, the MRI system 105 generating the plurality of echo-shifting gradients includes the MRI system 105 generating a first echo-shifting gradient with an area of (G−A) and thereby dephasing transverse magnetization from the first RF pulse, where A is the absolute value of the slice-refocusing gradient area (or one half of the slice-selection gradient area as shown in FIG. 4A) and G is the absolute value of the nominal dephasing/rephasing gradient area as detailed thereafter. After the transverse magnetization is dephased, the MRI system 105 generates the second RF pulse and thereby excites the stored longitudinal magnetization. After the second RF pulse is generated, the MRI system 105 generates the second echo-shifting gradient with an area of −G and thereby dephases transverse magnetization from the second RF pulse and rephases a signal produced by the first RF pulse. And after the signal produced by the first RF pulse is acquired, the MRI system generates the third echo-shifting gradient with an area of G and thereby dephases transverse magnetization from the first RF pulse and rephases a signal produced by the second RF pulse. In a further optional implementation, G′=G−A with A being the absolute value of the area of a slice-refocusing gradient associate with the first or the second RF pulse.

In one optional implementation, the non-transitory computer-readable medium includes the first echo-train with blip-up phase-encoding acquiring the rephased signal produced by the first RF pulse.

In another optional implementation, generating the plurality of echo-shifting gradients further includes, after the first echo-train is generated, the MRI system 105 generating a third echo-shifting gradient with an area of G and thereby rephasing a signal produced by the second RF pulse and dephasing the signal produced by the first RF pulse.

In a further optional implementation, the non-transitory computer-readable medium includes the second echo-train with blip-down phase-encoding acquiring the rephased signal produced by the second RF pulse.

In still another optional implementation, the esEPI-BUDA pulse sequence includes, after acquiring the rephased signal produced by the first RF pulse and before acquiring the rephased signal produced by the second RF pulse, the MRI system 105 generating a gradient having one half (½) of an individual phase-encoding blip gradient area (G_{y}).

In one optional implementation, the MRI system 105 conducting joint image reconstruction based on the two acquired k-space datasets includes the MRI system generating dynamic maps of a main magnetic field. Then the MRI system 105 incorporates the dynamic maps of a main magnetic field into a forward joint parallel imaging reconstruction model with Hankel structured low-rank constraints and thereby corrects the image geometric distortion.

In one optional implementation, the MRI system 105 conducting joint image reconstruction based on the two acquired k-space datasets includes the MRI system 105 combining the two acquired k-space datasets and thereby improving the image signal-to-noise ratio.

### V. Example 1

### Materials and Methods

### 3D ESEPI-BUDA Sequence

FIGS. 4A-B show an example of the 3D esEPI-BUDA pulse sequence and its corresponding k-space trajectories. 3D esEPI-BUDA uses two RF pulses to acquire the blip-up and blip-down datasets in a single TR (or shot). The two signals from the two RF pulses are time-shifted and individually selected by three dephasing/rephasing gradients (or echo-shifting gradients; (G−A), −G, and G) applied along the slab-selection direction (G_{z}). The first echo-shifting gradient with an area of (G−A) acts as a spoiler to dephase the transverse magnetization shortly after the first RF pulse α. (Note that A is the net area of slab-refocusing gradient associated with the first RF pulse.) After the transverse magnetization is dephased, the second RF pulse β is applied to excite the stored longitudinal magnetization, followed by the second echo-shifting gradient with an area of −G. This gradient dephases the transverse magnetization from RF pulse β, while, together with the net slab-selection gradient area (2A-A=A) associated with RF pulse β, rephasing the transverse magnetization produced by RF pulse α.

The rephased signal is acquired by the first EPI echo-train with blip-up phase-encoding (black). The third and final echo-shifting gradient with an area of G is placed after the first readout echo-train to rephase the signal produced by RF pulse β, while dephasing the remaining transverse magnetization from RF pulse α. The rephased signal is acquired by the second echo-train with blip-down phase-encoding (gray). k-Space data from both echo-trains are under-sampled (e.g., by two-fold) to shorten the echo-train length, thus enabling short and consistent TEs (e.g., 30 ms for fMRI at 3T) in both acquisitions. To make the echo times of the two echo-trains the same, the time delay between the two RF pulses (α and β) is determined by the center-to-center length of the two echo-trains (i.e., the summation of the third echo-shifting gradient width plus the duration of an individual echo-train).

The signal in the first echo-train is attenuated by cos² (β/2), and thus is proportional to M₀·sin α· cos² (β/2), where the M₀ is the longitudinal magnetization. The signal in the second echo-train is proportional to M₀·cos α·sin β, provided that the time delay (˜20 ms) between the two RF pulses is short as compared to the T1 value of the tissues. To equalize the signals for the two echo-trains, the flip angles α and β need to satisfy the following condition:

$$sin α \cdot {cos}^{2} ( β / 2 ) = cos α \cdot sin β$$

The esEPI-BUDA sequence also contains a small gradient with ½ phase-encoding blip area (G_{y}) prior to the second echo-train so that the two k-space trajectories can be interleaved (FIG. 4B). This allows the two k-space datasets to be effectively used in the Hankel structured low-rank image reconstruction. In principle, the esEPI-BUDA pulse sequence described above can be used for 2D or 3D imaging. In the former case, the slab-selection gradient along the G_{z}-axis is reduced to a slice-selection gradient, while in the latter case, a conventional stepping phase gradient is applied along the G_{z}-axis, providing spatial localization for both echo trains as shown in FIG. 4A.

### Imaging Experiments:

For fMRI applications, a 3D version of esEPI-BUDA was implemented on a GE MR750 3T scanner (GE Healthcare, Waukesha, Wisconsin, USA) to avoid the excessive SNR penalty that would incur in 2D implementations. Phantom and human in vivo experiments were performed using a 32-channel head coil (Nova Medical, Inc., Wilmington, Massachusetts, USA) to demonstrate the proposed esEPI-BUDA technique.

In the phantom experiment, a GE DQA (Daily Quality Assurance) phantom was used to validate the pulse sequence and its associated 3D image reconstruction. Phantom images from the 3D esEPI-BUDA sequence were acquired with the following parameters: TR/TE=100/40 ms, volume TR (TRᵥₒₗ)=3.2 s (where the TRᵥₒₗ is defined as the time taken to acquire a 3D volume), flip angles: α≈β=15°, FOV=180×180×128 mm³, acquisition matrix=72×72×32, spatial resolution=2.5×2.5×4.0 mm³, under-sampling factor along the in-slab phase-encoding direction=2 (i.e., the length of each echo-train (“ETL”)=36; total length of two echo-trains=72), and echo spacing=0.528 ms. The corresponding k-space data from the two echo-trains were separately reconstructed using SENSE, followed by joint image reconstruction (see Image reconstruction). For comparison, images over the same volume were also acquired using 3D fully-sampled EPI (flip angle=15°) with blip-up and blip-down acquisitions separately. To establish a reference to assess the improvement in image distortion reduction, additional images were obtained using a conventional 3D fast SPGR sequence (TR/TE=10.6/1.4 ms, flip angle=10°, FOV=180×180×128 mm³, and acquisition matrix=72×72× 32).

The in vivo experiment aimed at demonstrating the 3D esEPI-BUDA sequence for fMRI with visual stimulation. The imaging parameters were: TR/TE=75/30 ms, TRᵥₒₗ=2.4 s, α≈β=15°, FOV=220×220×128 mm³, acquisition matrix=72×72×32, spatial resolution=3.1×3.1× 4.0 mm³, under-sampling factor along the in-slab phase-encoding direction=2, and echo spacing=0.456 ms. For comparison, images over the same volume were also acquired using 3D fully-sampled EPI (flip angle=) 15° with blip-up and blip-down acquisitions separately. Similar to the phantom experiment, a conventional 3D fast SPGR image with matched FOV was acquired as a reference (TR/TE=10.5/1.4 ms, flip angle=10°, FOV=220×220× 128 mm³, and acquisition matrix=72×72× 32).

For the in vivo experiment, visual stimulation was delivered using a commercial system (SensaVue, Invivo Corporation, Gainesville, Florida, USA) with a dark-gray and light-gray checkboard pattern flashing at 8 Hz. This block-design paradigm contained six 48 s blocks, each with a 24 s stimulation period and a 24 s rest. The total acquisition time was 4 min and 48 sec. Six healthy human subjects (33.2±5.9 years) were asked to fixate on the cross-hair presented at the center of the visual field during the experiment. The fMRI stimulation delivery system was integrated with an infrared camera focusing on the subject's pupil so that the subject's motion and attentiveness to the fMRI task were monitored in real time. The camera was positioned at a 90° angle with respect to the optical pathway of the visual stimulation light. A large hot mirror (35″×17″) was positioned at a 45° angle with respect to both the incident infrared and visible light beams, allowing 98% transmission of visible light while reflecting 97% of infrared.

### Image Reconstruction:

The raw k-space data acquired with the 3D esEPI-BUDA sequence were preprocessed before image reconstruction: First, phase correction was performed to remove the Nyquist ghosting artifacts. The zeroth- or first-order phase differences between the odd and even encodings of each echo-train were corrected based on a reference scan by setting the amplitude of the phase-encoding gradients to zero. Second, to speed up the image reconstruction, a model based on the principal component analysis (“PCA”) was used to linearly concatenate the raw data from 32 channels into 16 channels. Coil sensitivity profile was estimated using the ESPIRIT approach with the data from the FOV-matched distortion-free 3D SPGR sequence. After the preprocessing, distortion-corrected images were reconstructed from the k-space data with the pipeline described in the following paragraphs.

FIG. 5 shows the steps involved in 3D esEPI-BUDA image reconstruction. Each under-sampled echo-train dataset (i.e., blip-up and blip-down) first underwent 3D SENSE reconstruction individually using the following equation:

$$\tilde{I} = {arg}_{I} min {‖ UF ( SI ) - d ‖}_{2}^{2}$$

where U is the sampling mask of k-space locations, F represents the Fourier transform operator, S is the coil sensitivity, d is the k-space dataset, I is the 3D SENSE-reconstructed image, and Ĩ is the resultant I after iterations. A 3D projection onto convex sets (“POCS”) algorithm was employed to solve the above equation.

After obtaining the two 3D SENSE images with blip-up and blip-down encoding individually, TOPUP in FSL was used to estimate a B₀-field map E, which was subsequently incorporated to jointly reconstruct the data from both echo-trains, as follows:

$$\tilde{I} = arg {min}_{I} {\sum }_{t = 1}^{2} {‖ {U}_{t} {F}_{t} ( {ESI}_{t} ) - {d}_{t} ‖}_{2}^{2} + λ {‖ ℋ ( I ) ‖}_{*}$$

where t is the echo-train index (1 or 2), Uₜ is the sampling mask of t^{th} echo-train, Fₜ is the fast Fourier transform operator for i^{th} under-sampled echo-train, E is the B₀-field map estimated using TOPUP in FSL, as shown in FIG. 5, S is the coil sensitivity information, Iₜ is the target distortion-corrected image, and dₜ is the acquired under-sampled k-space data of t^{th} echo-train. [symbol] (I) represents the Hankel low-rank matrix which enforces low-rankness among different echo-trains, ∥·∥, denotes the nuclear norm of the matrix, which is the sum of singular values, and A is the parameter to tune the weight of structured low-rank regularization.

In the 3D esEPI-BUDA joint image reconstruction, the Hankel structured low-rank constraint mitigated the phase errors between the two echo-trains and background noise based on the hypothesis that the k-space data of MR images typically have limited spatial support and/or slowly varying phase. Herein, the Hankel matrix was constructed by consecutively picking up 9×9×9 neighborhood points in k-space from each echo-train as a Hankel-block, followed by concatenating them in the column dimension. A POCS-like approach was used to solve this equation. In the POCS-iterative framework, root-mean-square error of less than 1% between two successive iterations was chosen to indicate convergency.

All image reconstructions were implemented in MATLAB (R2019b; the Mathworks, Natick, MA, USA) for off-line reconstruction on a Linux server (CentOS, Intel® Core™ i9-7920X CPU @ 2.90 GHz and 128 GB RAM).

### Data Analysis:

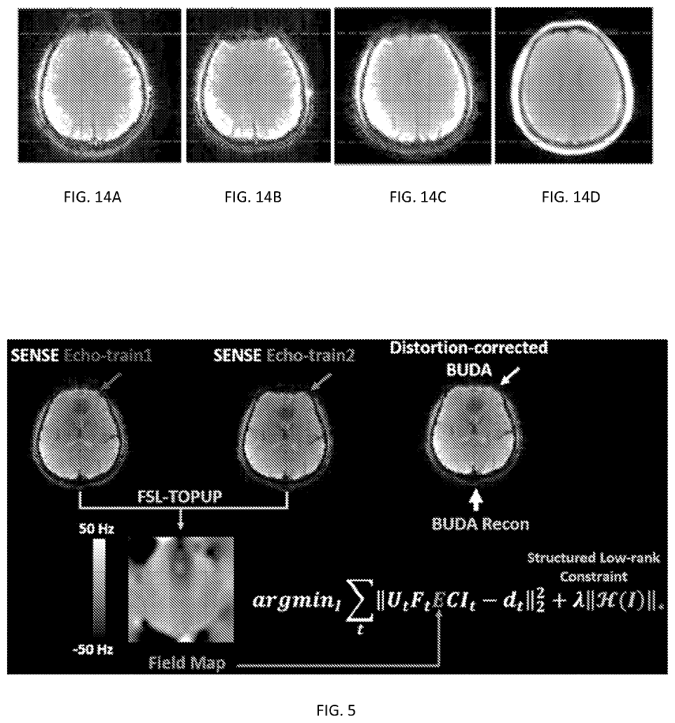

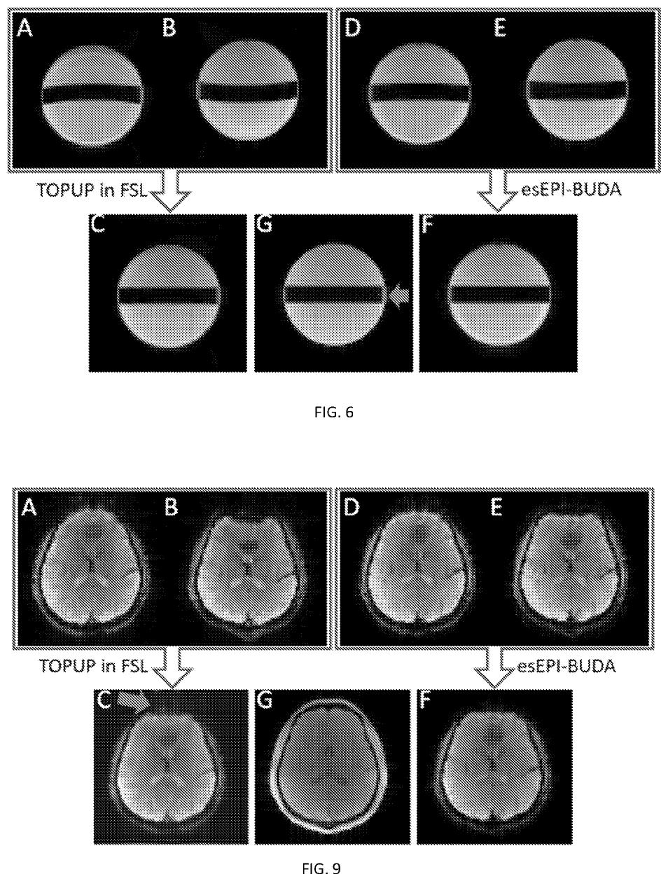

To demonstrate the performance of 3D esEPI-BUDA in terms of data acquisition efficiency and distortion correction effectiveness, images from the DQA phantom and fMRI experiments were evaluated and compared between 3D esEPI-BUDA and a conventional approach with separate blip-up and blip-down acquisitions followed by a correction method using TOPUP in FSL. This method (called 3D EPI TOPUP thereafter) performs image-domain registration between the two separately acquired datasets, each with 3D full sampling. In the DQA phantom experiment, the horizontal low-signal block (as indicated by the arrow in image G of FIG. 6) serves as a fiducial mark to qualitatively assess the effectiveness of geometric distortion correction produced by the 3D esEPI-BUDA with joint reconstruction and the two separate blip-up/down acquisitions with 3D EPI TOPUP, respectively. In the comparison, an image acquired by the 3D SPGR sequence was used as a reference. Three quantitative image analyses were also performed, including structural similarity (SSIM), normalized root-mean-square error (NRMSE), and SNR to compare the image quality.

In the process of image reconstruction of fMRI data, B₀-field maps (E) were estimated at each time point (i.e., TRᵥₒₗ) as shown in the foregoing equation. The mean value and the standard deviation of E across the time points were calculated to evaluate the time evolution of B₀-field maps. The dynamic change of the B₀-field map was analyzed at the frontal and occipital lobes. The resulting real-time spatial dislocation could be calculated by:

$$Δ d = \frac{2 π Δ f}{BW \cdot Δ k}$$

where Δf is the amount of off-resonance in Hertz that can be derived from the B₀-field map, Δk is the k-space sampling interval, and BW is the sampling bandwidth along the blipped phase-encoding direction, which is inversely related to the echo spacing. Δd was used to quantify the geometric distortion at each point in the fMRI experiment.

fMRI data were analyzed using SPM8 on MATLAB. Motion correction and spatial smoothing (FWHM=6 mm) were applied to magnitude images, followed by statistical analyses using a general linear model for activation detection with a P-value threshold (FWE corrected) of <0.05 and a spatial cluster size of at least 30 pixels. The MarsBar toolbox was used to extract and analyze the time courses. Averaged time courses with standard deviations across the subjects were compared among 3D esEPI-BUDA, the two separately acquired datasets with blip-up and blip-down prior to distortion correction, and distortion-corrected images using 3D EPI TOPUP.

### Results

FIG. 6 displays images from the phantom experiment, including 3D fully-sampled EPI with separate blip-up (image A) and blip-down (image B) acquisitions, the corresponding 3D EPI TOPUP (image C), individually SENSE-reconstructed images from the first (image D) and second (image E) echo train of the 3D esEPI-BUDA sequence, a resultant esEPI-BUDA image (F), and a conventional 3D SPRG (image G) as a reference. For each display, a representative image corresponding to slice No. 16 of the DQA phantom was selected from the 3D volumetric dataset. The horizontal black block in the middle of the phantom (see the arrow in image (G) of FIG. 6) was bent upwards or downwards in (A), (B), (D), and (E). Comparison of the first-row images in FIG. 6 indicates that the sEPI-BUDA sequence produced high quality images (D) and € despite a two-fold scan time reduction as compared with the separate acquisition strategy for images (A) and (B). Images (D) and (E) exhibited less distortion (see the central block in the phantom) because of the use of parallel imaging. The second row of images show that both 3D EPI TOPUP in FSL and the joint k-space reconstruction employed in 3D esEPI-BUDA effectively corrected the image distortion with similar performance. Using the distortion-free SPGR image in image (G) of FIG. 6 as a reference, the esEPI-BUDA image (F) of FIG. 6 exhibited a higher degree of similarity with an SSIM of 0.91 and an NRMSE of 0.06 than the image reconstructed from two separate acquisitions (image (C) of FIG. 6; SSIM=0.87; NRMSE=0.08). Quantitative measurement of SNR in the phantom revealed an approximately 42.2% increase in the 3D esEPI-BUDA image (image F of FIG. 6; SNR=86.4) compared with the individual 3D SENSE-reconstructed images (images (D) and (E) of FIG. 6; SNR=60.8). However, the SNR decreased by approximately 9.3% and 6.0% compared with the 3D fully-sampled EPI images (images (A) and (B) of FIG. 6; SNR=95.3) and the resultant EPI TOPUP image (image (C) of FIG. 6; SNR=91.9), respectively.

The performance of the distortion correction on a representative human subject (26-year-old male) is demonstrated in FIGS. 8A-C. Compared with the SENSE-reconstructed EPI images using the two individual echo trains (FIGS. 8A and 8B), esEPI-BUDA reduced the degree of image distortion and increased the SNR (FIG. 8C). This becomes more apparent by analyzing a representative section selected from the 3D volume (FIG. 9). Similar to the phantom results in FIG. 6, each echo train in esEPI-BUDA produced good image quality as compared to the images acquired separately with the doubled scan time (first row of images in FIG. 9). Compared to the images (A) and (B) in FIG. 9, the reduced image distortion (e.g., in the frontal area) in images (D) and (E) in FIG. 9 was attributed to the use of parallel imaging in esEPI-BUDA in an attempt to shorten the individual echo-train length. Both 3D EPI TOPUP on the individually acquired images and joint reconstruction employed in esEPI-BUDA effectively corrected the dilation and compression artifacts of the images (C) and (F) of FIG. 9, when a conventional SPGR image was used as a reference (image (G) of FIG. 9).

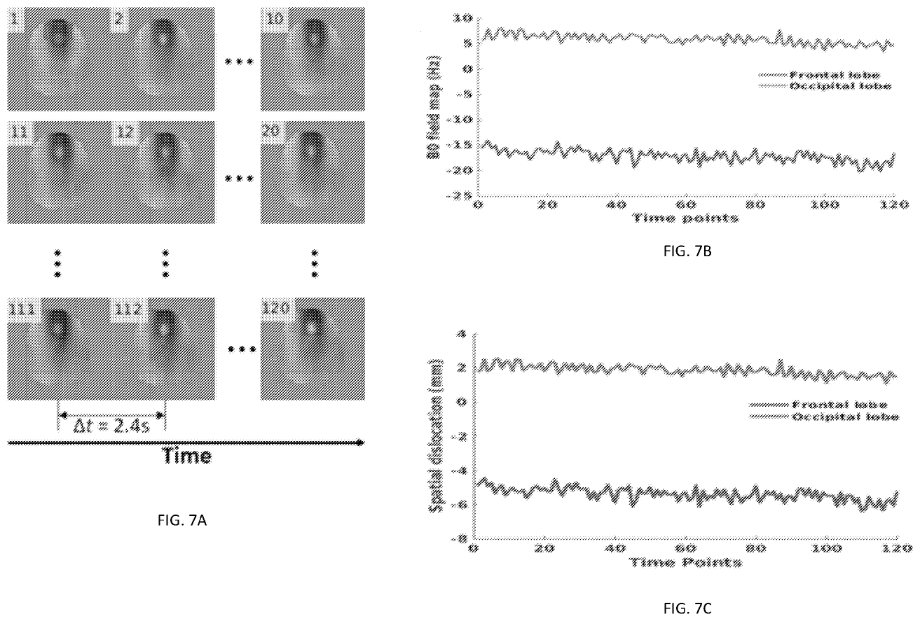

FIG. 7A shows the dynamic B₀ field maps in the fMRI experiment obtained using 3D esEPI-BUDA. The time evolution of the B₀ field maps was captured with a temporal resolution of 2.4 s (i.e., volume TR). Each B₀ field map corresponded to a specific time point in the fMRI scan (numbered by 1, 2, . . . , 120), and the collection of the images spanned a duration of 4 min and 48 sec. Representative B₀ field evolutions obtained from the frontal and occipital lobes are displayed in FIG. 7B. The B₀-field values (mean±standard deviation) in the two areas were-17.1±1.2 Hz and 5.9±1.0 Hz, respectively. During the entire time course, the maximal B₀-field difference (maximum value-minimum value) was approximately 6.2 Hz and 4.7 Hz for the frontal and occipital lobes, respectively. The resulting variations of spatial dislocation Δd were estimated to be 1.95 mm and 1.47 mm (FIG. 7C), respectively. These were approximately one half of the voxel size, indicating the necessity of obtaining the B₀-field map at each time point to dynamically correct the geometric distortion in an fMRI scan.

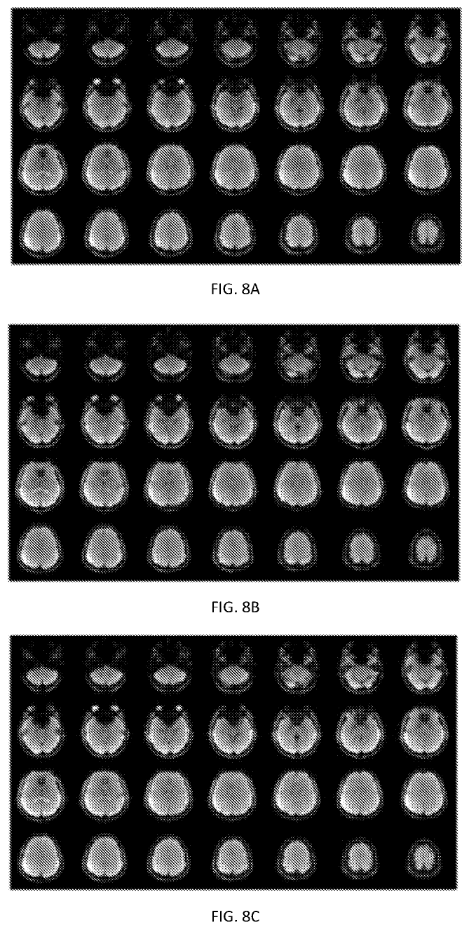

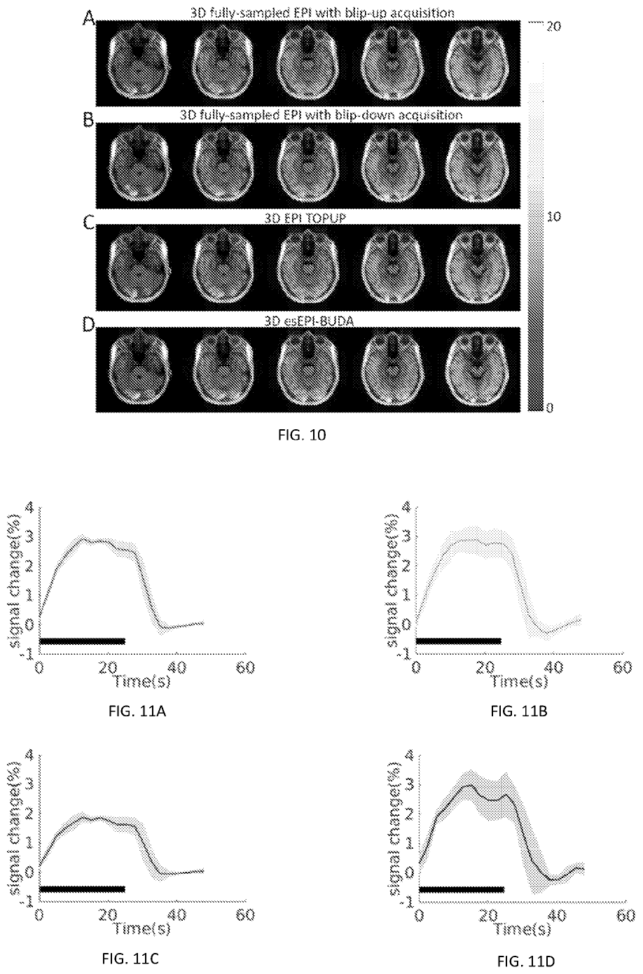

Results from the visual fMRI experiments are illustrated in FIG. 10, where five contiguous activation maps are overlaid on the corresponding T1-weighted images. fMRI activations were observed in the visual cortex, as expected. The activation maps of 3D esEPI-BUDA co-registered better with the T1-weighted structure images than the two separately acquired 3D EPI images and their resultant 3D EPI TOPUP images in which the activations were beyond the border of brain parenchyma. This illustrates the advantage of esEPI-BUDA, which is capable of dynamically correcting the distortions during the fMRI time course, over the conventional approach. Comparable average activated volumes across subjects were detected (separate blip-up acquisition: 30.6 cm³, separate blip-down acquisition: 25.3 cm³, 3D EPI TOPUP: 30.7 cm³, and 3D esEPI-BUDA: 27.2 cm³). The average time courses across the six subjects at the visual cortex are shown in FIGS. 11A-D, where the 3D esEPI-BUDA sequence and the sequences with separate acquisitions produced similar BOLD signal changes (˜3%), which was higher than 3D EPI TOPUP images (˜2%). The decreased signal change in 3D EPI TOPUP was likely caused by the data interpolation in the TOPUP tool of FSL. The consistency in both the average activated volume and the BOLD signal change indicates the potential of using the 3D esEPI-BUDA sequence for BOLD activation detection.

As set forth herein, an exemplary esEPI-BUDA technique has been demonstrated, in which two interleaved k-space datasets with reversed k-space trajectories can be acquired in a single shot. Compared to traditional BUDA techniques for image distortion correction, esEPI-BUDA offers a major advantage by halving the scan times. With simultaneous blip-up and blip-down acquisitions in a single shot, esEPI-BUDA can also be more resilient to motion and allow B₀-field maps to be estimated dynamically throughout a time series. The latter is particularly desirable as the B₀-field maps can be incorporated into the forward joint parallel imaging reconstruction model with Hankel structured low-rank constraint to correct the geometric distortion.

As an attractive technique for distortion correction in EPI, BUDA has been successfully applied to susceptibility-weighted imaging, diffusion-weighted imaging, and T2 mapping. Extension to fMRI has been challenging as the acquisitions of blip-up and blip-down data in two separated TRs lead to a substantially degraded temporal resolution (i.e., >4 s). By utilizing the echo-shifting strategy, esEPI-BUDA incorporates two echo-trains into a single pulse sequence, overcoming the temporal resolution limitation as demonstrated by the short volume TR (e.g., 2.4 s) achieved in fMRI experiments.

Owing to the pair-wise acquisition of the blip-up and blip-down datasets, esEPI-BUDA is capable of capturing real-time B₀-field temporal variations in an fMRI scan. The temporal variations can be a reflection of subject motion, physiological respiration, heating of the gradient system, mechanic vibration, and/or other system instabilities. Although TOPUP in FSL is effective for correcting distortion at a single time-point (image (C) in both FIGS. 6 and 9), distortion correction based on retrospective techniques to estimate the B₀-field map cannot account for the dynamic off-resonance effects. In contrast, esEPI-BUDA is capable of monitoring the dynamic B₀-field change and using that information to correct image distortion at each time point (FIGS. 7A-C and 10). This can be particularly useful for fMRI, diffusion imaging, and arterial spin labeling whose scan times can last for several minutes or longer.

In parallel to the two-fold scan time reduction by using the echo-shifting strategy, conventional parallel imaging was also employed in the G_{y}-direction with an acceleration factor of two to shorten the echo-train length for the individual blip-up and blip-down acquisitions. The use of parallel imaging also reduced the image distortion (images (A) and (B) of FIG. 9 vs. images (D) and (E) of FIG. 9) as a result of an increased effective bandwidth along the blipped phase-encoding direction. Higher acceleration factors can be potentially achieved by applying data under-sampling in both k_{z} and k_{y} directions to reduce not only the echo-train length but also the volume TR. An example is to employ 2D CAIPIRINHA (controlled aliasing in parallel imaging results in higher acceleration) to accomplish multi-dimensional acceleration. The feasibility of using higher acceleration factors and temporal resolutions in esEPI-BUDA will be further explored in future studies.

In the esEPI-BUDA sequence, the blip-up and blip-down echo-trains were interleaved so that the complete k-space datasets could be used in the joint reconstruction. The joint reconstruction can significantly reduce the g-factor noise penalty when compared with reconstructions on the blip-up and blip-down acquisitions separately. In this study, Hankel structured low-rank regularization was also exploited to strengthen the joint of the blip-up and blip-down datasets by a low-rank constraint, which not only reduces the g-factor, but also removes the phase errors between different echo-trains without the need of navigation. The echo-shifting strategy results in a theoretical SNR loss of (1−cos² (β/2)). For the phantom studies, the theoretical SNR loss was only ˜1.7% because of a low flip angle (β=15°). The experimentally measured SNR loss (9.3%), however, was higher, likely caused by the noise amplification (g-factor) during image reconstruction.

This example study has limitations. First, 3D fMRI was employed as an example to illustrate the esEPI-BUDA technique. Although the concept of esEPI-BUDA can be applied to both 2D and 3D acquisitions, esEPI-BUDA for 2D multi-slice fMRI would require a large flip angle of the RF excitation pulse. With a large flip angle, however, the SNR loss due to the use of the echo-shifting strategy can be substantial. For example, with optimal flip angles of α≃47° and βδ56°, the SNR in 2D esEPI-BUDA would be reduced by ˜50% from that of a conventional 2D EPI sequence with a 90° RF excitation pulse for excitation. Performing 2D esEPI-BUDA acquisitions at a higher magnetic field (e.g., 7 Tesla) may compensate for such SNR loss. Second, the echo-shifting strategy limits the shortest TE achievable in esEPI-BUDA. Assuming that partial Fourier k-space encoding is not employed, the shortest TE is given by 0.5×Tₛₛ+2×Tₑₛ+1.5×esp×ETL, where Tₛₛ is the duration of slab-selection gradient, Tₑₛ is the duration of the echo-shifting gradient, esp is the echo spacing in the echo-train, and ETL is the echo-train length of each echo-train. The analysis showed that the minimum TE of ˜28 ms could be achieved in the visual fMRI experiment, which was fortunately adequate for BOLD contrast. A lengthy echo-train needed by high spatial resolution, however, can increase the minimum TE in the 3D esEPI-BUDA sequence. An excessively long TE reduces the SNR and increases the sensitivity to magnetic field inhomogeneities and flow effects. The issue can be mitigated by reducing the duration of the echo-shifting gradients and/or incorporating parallel imaging with a higher acceleration factor to shorten the echo-trains. The latter, however, can decrease the SNR, exacerbate residual aliasing, and compromise BOLD detectability.

As set forth herein, an exemplary technique, esEPI-BUDA, has been demonstrated to enable 3D distortion-corrected whole-brain 3D fMRI without increasing the scan time. The method integrates the blip-up and blip-down data acquisitions in a single shot, followed by joint reconstruction. The integrated data acquisition strategy also produces time resolved B₀-field maps that can be incorporated into image reconstruction to achieve dynamic image distortion correction. The method has been demonstrated on the phantom and human brain. Distortion-corrected 3D echo-planar images were successfully obtained with adequate SNR and BOLD sensitivity. With these demonstrations, esEPI-BUDA methods are expected to benefit other neuroimaging applications such as 3D/2D fMRI, diffusion imaging, and perfusion imaging.

### VI. Example 2

### Methods

3D esEPI-BUDA:

FIGS. 4A-B show the principle of 3D esEPI-BUDA pulse sequence and its corresponding k-space trajectories. 3D esEPI-BUDA uses two RF pulses to acquire the blip-up and blip-down datasets in a single TR (or shot). The two signals from the two RF pulses are time-shifted and individually selected by three dephasing/rephasing gradients (or echo-shifting gradients; (G−A), −G, and G) applied along the slab-selection direction (G_{z}). The first echo-shifting gradient with an area of (G−A) acts as a spoiler to dephase the transverse magnetization shortly after the first RF pulse a. After the transverse magnetization is dephased, the second RF pulse β is applied to excite the stored longitudinal magnetization, followed by the second echo-shifting gradient with an area of −G. This gradient dephases the transverse magnetization from RF pulse β, while rephasing the transverse magnetization produced by RF pulse a. The rephased signal is acquired by the first EPI echo-train with blip-up phase-encoding (black). The final echo-shifting gradient with an area of G is placed after the first readout echo-train to rephase the signal produced by RF pulse β, while dephasing the signal from RF pulse α. The rephased signal is acquired by the second echo-train with blip-down phase-encoding (gray). k-Space data from both echo-trains are under-sampled (e.g., by two-fold) to shorten the echo-train length, thus enabling short and consistent TEs (e.g., 30 ms for fMRI at 3T) for both acquisitions. To equalize the signals for the two echo-trains, the flip angles α and β need to satisfy the following condition:

$$sin α \cdot {cos}^{2} ( β / 2 ) = cos α \cdot sin β$$

The esEPI-BUDA sequence also contains a small gradient with ½ phase-encoding blip area (G_{y}) prior to the second echo-train so that the two k-space trajectories are interleaved (FIG. 4B) for effective joint reconstruction.

### Buda Reconstruction:

FIG. 12 shows the steps involved in 3D esEPI-BUDA image reconstruction. Each echo-train dataset first underwent SENSE reconstruction using TOPUP in FSL to estimate a field map E, which was subsequently incorporated to jointly reconstruct the data from both echo-trains according to:

$$arg {min}_{I} {\sum }_{t} {‖ {F}_{t} {ECI}_{t} - {d}_{t} ‖}_{2}^{2} + λ {‖ ℋ ( I ) ‖}_{*}$$

where t is the echo-train index (1 or 2), Fₜ is the Fourier operator, C is the coil sensitivity, and Iₜ and dₜ are the targeted distortion-corrected image and the k-space data for the t^{th} echo-train, respectively. The constraint ∥ [symbol] (I)∥. enforces low-rank prior on the block-Hankel representation of the two datasets, and λ is the parameter to tune the weight of structured low rank regularization.

### Experiments

The 3D esEPI-BUDA sequence was implemented on a GE MR750 3T scanner. 3D fMRI experiments were performed on healthy human brains using a 32-channel head coil with a visual stimulation paradigm. The paradigm contained six 48-s blocks with 24-s stimulus followed by 24-s rest. The imaging parameters were: TR/TE=75/30 ms, volume TR=2.4s, α≈β=15° (Ernst angle), FOV=220×220×128 mm³, acquisition matrix=72×72×32, spatial resolution=3.1×3.1×4.0 mm³, and under-sampling factor along the in-slab phase-encoding direction=2 (i.e., the length of each echo-train=36; total length of two echo-trains=72). For comparison, images over the same volume were also acquired using 3D fully-sampled EPI (flip angle=) 15° with blip-up and blip-down acquisitions separately.

### Results

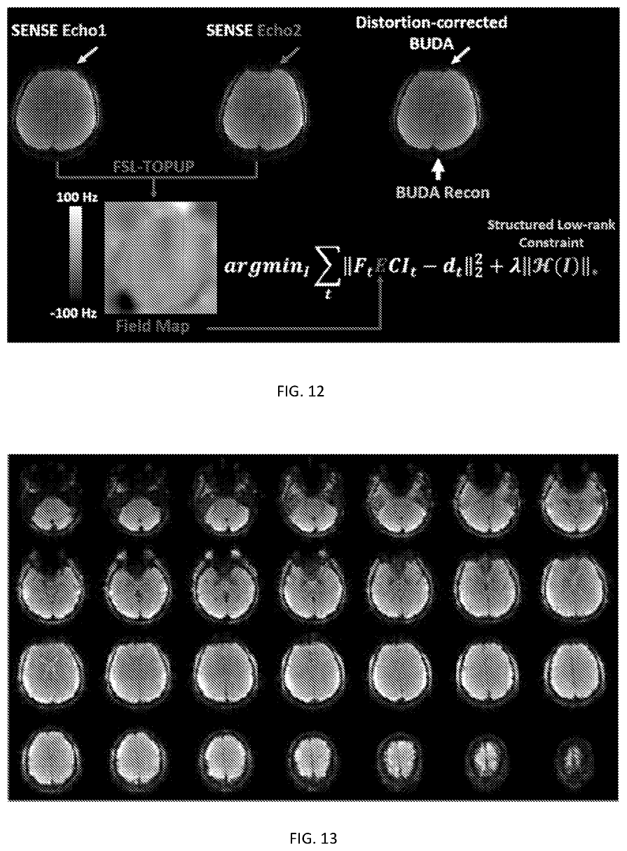

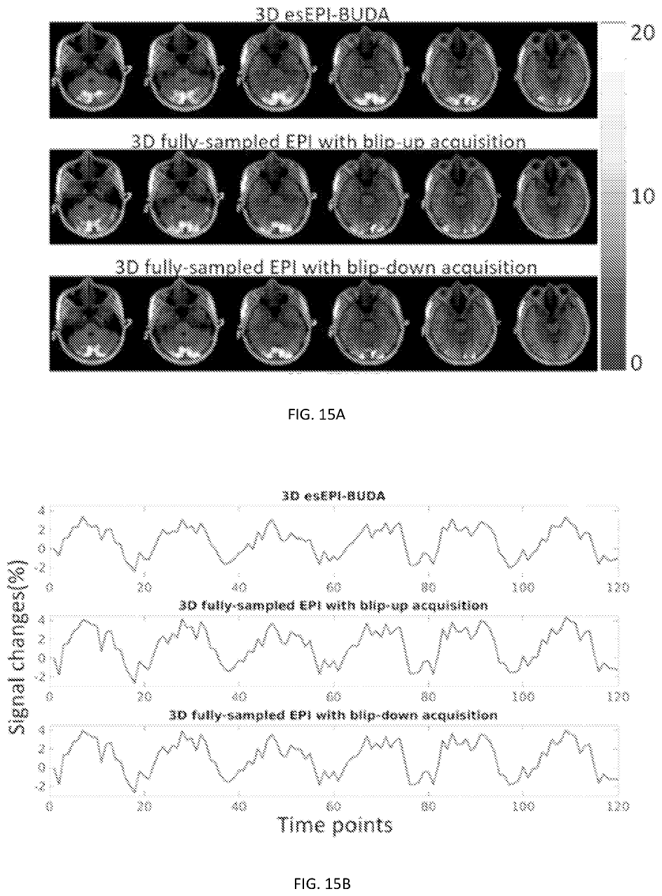

FIG. 13 displays a set of representative whole-brain images obtained using 3D esEPI-BUDA, illustrating excellent image quality. The BUDA reconstruction produced images with noticeably reduced geometric distortion, especially at the frontal lobe, when compared to the 3D fully-sampled EPI images (FIG. 14A-D). Results from the fMRI experiment are illustrated in FIG. 15A where six contiguous activation maps from 3D esEPI-BUDA are overlaid on the corresponding T1-weighted images. fMRI activations were observed in the visual cortex, as expected. FIG. 15B shows the time evolution of the BOLD signal change (˜4.0%), which is comparable with that of 3D fully-sampled EPI.

An exemplary pulse sequence-3D esEPI-BUDA has been demonstrated herein that accomplished distortion correction with 3D whole-brain coverage by acquiring the blip-up and blip-down datasets in a single shot without increasing the scan time and without being subject to inter-shot motion. Although the demonstration was for 3D fMRI, the same strategy can be extended to other EPI-based applications such as 2D fMRI, 2D/3D diffusion, and 2D/3D perfusion imaging. Also, while the above description uses fMRI as an example, the methods described herein can be applied for diffusion imaging, perfusion imaging, and any other MRI applications where echo-train-based echo planar imaging acquisitions are employed. Other embodiments of systems, methods and components constructed in accordance with the principles herein are contemplated as well.

1. A method for using an MRI system electrically coupled to a computing device, the method comprising:
generating, via the MRI system, an echo-shifted echo-planar imaging with blip up/down acquisition (“esEPI-BUDA”) pulse sequence comprising a first radiofrequency (“RF”) pulse and a second RF pulse, the first RF pulse followed by a first echo-train that is interleaved with the first and the second RF pulses, and the second RF pulse followed by a second echo-train such that the first and the second echo-trains have opposite phase-encoding blip gradient polarities to traverse echo planar imaging (“EPI”) k-space in a reversed order;
in response to the pulse sequence being generated, the MRI system acquiring two k-space datasets within a single shot; and
correcting image distortion, via the MRI system, based on the two acquired k-space datasets.

2. The method of claim 1, wherein the first RF pulse has a flip angle of a and the second RF pulse has a flip angle of β, with α and β satisfying the following condition:
$$sin α \cdot {cos}^{2} ( β / 2 ) = cos α \cdot sin β .$$

3. The method of claim 1, wherein generating the esEPI-BUDA pulse sequence further comprises:
generating, via the MRI system, a plurality of echo-shifting gradients applied along a direction perpendicular to the imaging plane.

4. The method of claim 3, wherein generating the plurality of echo-shifting gradients comprises:
generating, via the MRI system, a first echo-shifting gradient with an area of G′ and thereby dephasing transverse magnetization from the first RF pulse;
after the transverse magnetization is dephased, generating, via the MRI system, the second RF pulse and thereby exciting the stored longitudinal magnetization;
after the second RF pulse is generated, generating, via the MRI system, the second echo-shifting gradient with an area of −G and thereby dephasing transverse magnetization from the second RF pulse and rephasing a signal produced by the first RF pulse; and
after the signal produced by the first RF pulse is acquired, generating, via the MRI system, the third echo-shifting gradient with an area of G and thereby dephasing transverse magnetization from the first RF pulse and rephasing a signal produced by the second RF pulse.

5. The method of claim 4, where G′=G−A with A being the absolute value of the area of a slice-refocusing gradient associated with the first or the second RF pulse.

6. The method of claim 4, further comprising:
acquiring, via the first echo-train with blip-up phase-encoding, the rephased signal produced by the first RF pulse.

7. The method of claim 4, further comprising:
acquiring, via the second echo-train with blip-down phase-encoding, the rephased signal produced by the second RF pulse.

8. The method of claim 1, wherein the esEPI-BUDA pulse sequence further comprises:
after acquiring the rephased signal produced by the first RF pulse and before acquiring the rephased signal produced by the second RF pulse, generating, via the MRI system, a gradient having one half (½) of an individual phase-encoding blip gradient area (G_{y}).

9. The method of claim 1, further comprising:
under-sampling, via the MRI system, k-space data from the first echo-train and the second echo-train and thereby shortening a length of each of the first echo-train and the second echo-train.

10. The method of claim 1, wherein correcting image distortion based on the two acquired k-space datasets further comprises:
generating, via the MRI system, dynamic maps of a main magnetic field; and
incorporating, via the MRI system, the dynamic maps of the main magnetic field into a forward joint parallel imaging reconstruction model with Hankel structured low-rank constraints and thereby correcting image geometric distortion.

11. The method of claim 1, wherein correcting image distortion based on the two acquired k-space datasets further comprises:
combining, via the MRI system, the two acquired k-space datasets and thereby improving the image signal-to-noise ratio.

12. A non-transitory computer-readable medium having stored thereon program instructions that upon execution by a processor, cause performance of a set of steps comprising:
an MRI system generating an echo-shifted echo-planar imaging with blip up/down acquisition (“esEPI-BUDA”) pulse sequence comprising a first radiofrequency (“RF”) pulse and a second RF pulse, the first RF pulse followed by a first echo-train that is interleaved with the first and the second RF pulses, and the second RF pulse followed by a second echo-train such that the first and the second echo-trains have opposite phase-encoding blip gradient polarities to traverse echo planar imaging (“EPI”) k-space in a reversed order;
in response to the pulse sequence being generated, the MRI system acquiring two k-space datasets within a single shot; and
the MRI system correcting image distortion based on the two acquired k-space datasets.

13. The non-transitory computer-readable medium of claim 12, wherein the MRI system generating the esEPI-BUDA pulse sequence further comprises:
the MRI system generating a plurality of echo-shifting gradients applied along a direction perpendicular to the imaging plane.

14. The non-transitory computer-readable medium of claim 13, wherein the MRI system generating the plurality of echo-shifting gradients comprises:
the MRI system generating a first echo-shifting gradient with an area of G′ and thereby dephasing transverse magnetization from the first RF pulse;
after the transverse magnetization is dephased, the MRI system generating the second RF pulse and thereby exciting the stored longitudinal magnetization;
after the second RF pulse is generated, the MRI system generating the second echo-shifting gradient with an area of −G and thereby dephasing transverse magnetization from the second RF pulse and rephasing a signal produced by the first RF pulse; and
after the signal produced by the first RF pulse is acquired, the MRI system generating the third echo-shifting gradient with an area of G and thereby dephasing transverse magnetization from the first RF pulse and rephasing a signal produced by the second RF pulse.

15. The non-transitory computer-readable medium of claim 14, where G′=G−A with A being the absolute value of the area of a slice-refocusing gradient associated with the first or the second RF pulse.

16. The non-transitory computer-readable medium of claim 14, further comprising:
the first echo-train with blip-up phase-encoding acquiring the rephased signal produced by the first RF pulse.

17. The non-transitory computer-readable medium of claim 14, further comprising:
the second echo-train with blip-down phase-encoding acquiring the rephased signal produced by the second RF pulse.

18. The non-transitory computer-readable medium of claim 12, wherein the esEPI-BUDA pulse sequence further comprises:
after acquiring the rephased signal produced by the first RF pulse and before acquiring the rephased signal produced by the second RF pulse, the MRI system generating a gradient having one half (½) of an individual phase-encoding blip gradient area (G_{y}).

19. The non-transitory computer-readable medium of claim 12, wherein the MRI system correcting image distortion based on the two acquired k-space datasets further comprises:
the MRI system generating dynamic maps of a main magnetic field; and
the MRI system incorporating the dynamic maps of the main magnetic field into a forward joint parallel imaging reconstruction model with Hankel structured low-rank constraints and thereby correcting image geometric distortion.

20. The non-transitory computer-readable medium of claim 12, wherein the MRI system correcting image distortion based on the two acquired k-space datasets further comprises:
the MRI system combining the two acquired k-space datasets and thereby improving the image signal-to-noise ratio.
